(19) United States
(12) Patent Application Publication
(10) Pub. No.: US 2014/0379050 A1
(43) Pub. Date: Dec. 25, 2014

(54) LIGHT ACUPUNCTURE MODULE

(71) Applicant: GCSOL TECH CO., LTD., TAICHUNG (TW)

(72) Inventors: Jui-Fen PAI, Nantou County (TW); Tien-Jung HUANG, Taipei (TW); Fang-Chuan HO, Hsinchu (TW)

(73) Assignee: GCSOL TECH CO., LTD., TAICHUNG (TW)

(21) Appl. No.: 13/963,092

(22) Filed: Aug. 9, 2013

(30) Foreign Application Priority Data
Jun. 19, 2013 (TW) 102211474

(51) Int. Cl. A61N 5/06

(52) U.S. Cl. CPC A61N 5/0619
USPC 607/88

(57) ABSTRACT

A light acupuncture module includes a main member, a light emitting unit, and a driving unit. The main member has a touching face adapted to touch a human's skin. The light emitting unit is connected to the main member, and has a light emitting face, from which specific rays emits. The driving unit is electrically connected to the light emitting unit to supply the light emitting unit with electricity. Therefore, a user can get light acupuncture therapy while he/she attach the light acupuncture module on skin, and he/she can do anything he/she likes without any restriction.

The current application claims a foreign priority to the patent application of Taiwan No. 102211474 filed on Jun. 19, 2013.

### BACKGROUND OF THE INVENTION

1. Technical Field

The present invention relates to acupuncture, and more particularly to a light acupuncture module.

2. Description of Related Art

In traditional Chinese medicine (TCM), acupuncture is known around the world. The traditional acupuncture involves penetration of the skin with needles in order to stimulate certain acupuncture points on the body. However, every acupuncture point has its specific location and depth under the skin. Only a well-trained doctor can stimulate the acupuncture points right. Pain, needle phobia, and bruise are the common bad effects in acupuncture therapy.

In present days, light acupuncture therapy is invented, in which needle penetration is replaced by light. There are two types of devices for such light acupuncture therapy, fixed type and hand-held type.

The fixed type light acupuncture device has a box and a light emitter in the box to emit specific rays. The patient enters the box, and let the rays of the light emitter stimulate certain acupuncture points. It is impossible for an ordinary person to buy a fixed type light acupuncture device for acupuncture therapy at home.

The hand-held type light acupuncture device usually has a hand piece to emit specific rays. A user may hold the hand piece to let the rays stimulate certain acupuncture point. However, it is impossible for one to precisely aim the hand piece at some acupuncture points, such as the acupuncture points on the back, therefore, he/she still needs assistance in some cases.

### BRIEF SUMMARY OF THE INVENTION

In view of the above, the primary objective of the present invention is to provide a light acupuncture module, which makes one is easier to get light acupuncture therapy treatment done by oneself without any restriction.

In order to achieve the objective of the present invention, a light acupuncture module includes a main member, a light emitting unit, and a driving unit. The main member has a touching face adapted to touch a human's skin. The light emitting unit is connected to the main member, and has a light emitting face, from which specific rays emits. The driving unit is electrically connected to the light emitting unit to supply the light emitting unit with electricity.

Therefore, a user can get light acupuncture therapy while he/she attach the light acupuncture module on skin, and he/she can do anything he/she likes without any restriction.

### BRIEF DESCRIPTION OF THE SEVERAL VIEWS OF THE DRAWINGS

The present invention will be best understood by referring to the following detailed description of some illustrative embodiments in conjunction with the accompanying drawings, in which

FIG. 1 is a perspective view of a first preferred embodiment of the present invention;

FIG. 2 is a sectional view of the first preferred embodiment of the present invention;

FIG. 3 is a sectional view of a second preferred embodiment of the present invention;

FIG. 4 is a sectional view of a third preferred embodiment of the present invention;

FIG. 5 is a perspective view, showing the main member of the base and the belt;

FIG. 6 is a perspective view of a fourth preferred embodiment of the present invention;

FIG. 7 is a perspective view, showing the main member of the belt;

FIG. 8 is a perspective view of a fifth preferred embodiment of the present invention; and

FIG. 9 is a sectional view of the fifth preferred embodiment of the present invention.

### DETAILED DESCRIPTION OF THE INVENTION

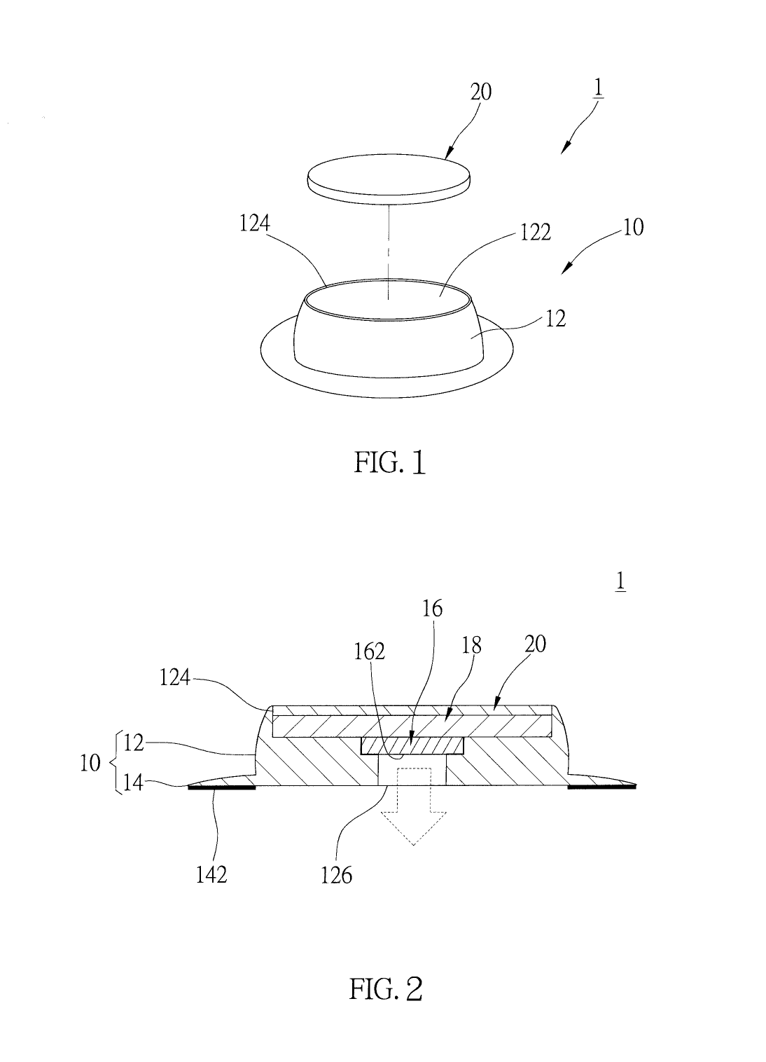

FIG. 1 and FIG. 2 show a light acupuncture module 1 of the first preferred embodiment of the present invention, which includes a main member 10, a light emitting unit 16, a driving unit 18, and a lid 20.

The main member 10 has a base 12 and a positioning device. In an embodiment, the positioning device is an adhesive layer 14. The base 12 has a room 122 therein, and a top opening 124 and a bottom opening 126 on a top and a bottom sides thereof to be communicated with the room 122. The adhesive layer 14 is provided on the bottom side of the base 12, and surrounds the bottom opening 126. The adhesive layer 14 has a touching face 142 adapted to be attached to a skin of human to affix the base 12 to a specific portion of the skin. In practice, the adhesive layer 14 is made of a material, such as silicon rubber, which has less chance to cause an allergy to skin. In an embodiment, the adhesive layer 14 is re-adherable. In an embodiment, the adhesive layer 14 is transparent, and covers the bottom opening 126 to isolate moisture.

The light emitting unit 16 is received in the room 122. In an embodiment, the light emitting unit 16 is a light emitting diode (LED), which has a light emitting face 162. The light emitting face 162 is under the bottom opening 126, and is kept a distance from the touching face 142. The bottom opening 126 will concentrate the rays of the LED, which is a point light source, so that the skin under the bottom opening 126 will be exposed under the rays. Besides, it may separate the light emitting unit 16 from skin in the light acupuncture therapy to avoid a scald of skin.

In another embodiment, the light emitting unit 16 is an organic light emitting diode (OLED), which is thinner and colder than LED, therefore, the distance from light emitting face 162 of the light emitting unit 16 to the touching face 142 could be shorter, or even let the OLED touch the skin.

The driving unit 18 includes a battery. The driving unit 18 is received in the room 122 of the base 12 through the top opening 124. The driving unit 18 supplies the light emitting unit 16 with electricity. In practice, the driving unit may be a button cell, a fuel cell, a Lithium battery, a thin film battery, or other small batteries.

The lid 20 is detachably mounted in the top opening 124 of the base 12. One may open the lid 20 to repair or replace the light emitting unit 16 and the driving unit 18. The lid 20 may be a plug to be squeezed into the top opening 124, or a bolt-like member to be screwed into the top opening 124.

One may attach the base 12 of the light acupuncture module 1 to a specific position on skin (acupuncture point) to let the rays of the light emitting unit 16 stimulate the acupuncture point under the bottom opening 126. The light acupuncture module 1 is attached to user's skin directly, and is hidden under clothe. Beside, no wire is necessary in the light acupuncture module 1, so that the user may get light acupuncture therapy anytime, anywhere by the light acupuncture module 1 of the present invention, and he/she may do anything he/she like in the therapy.

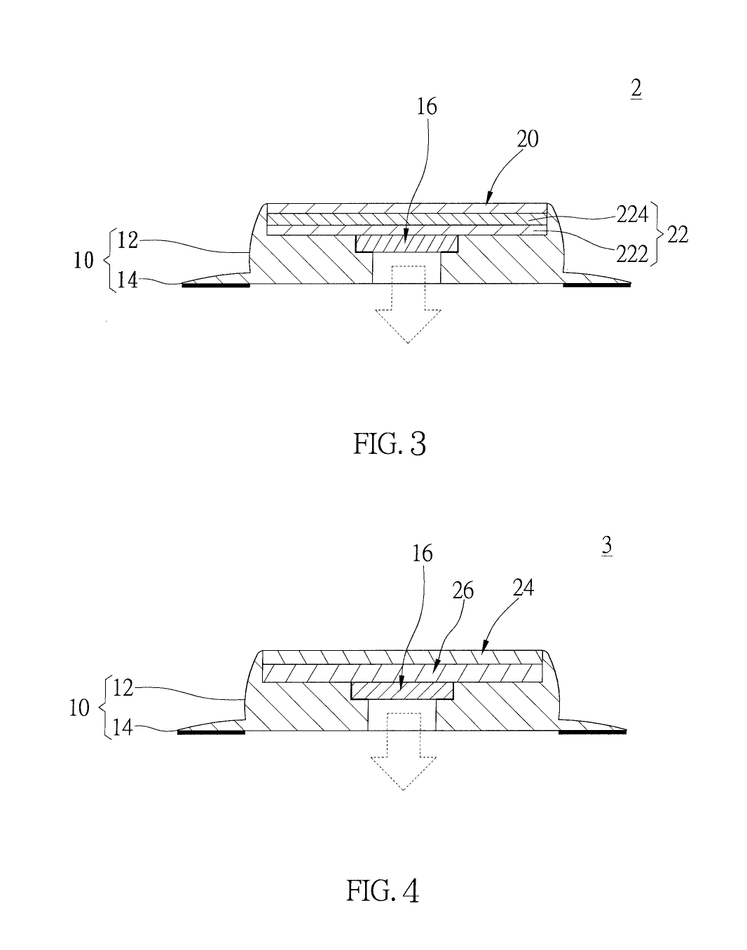

FIG. 3 shows a light acupuncture module 2 of the second preferred embodiment of the present invention, which is similar to the first embodiment, except that a driving unit 22 includes a control circuit 222 and a battery 224. The battery 224 is electrically connected to the control circuit 222, and the control circuit 222 is electrically connected to the light emitting unit 16. The control circuit 222 controls an intensity of the rays of the light emitting unit 16. In an embodiment, a plurality of switches (not shown) are connected to the control circuit 222 for manipulation to adjusting the intensity.

In an embodiment, the light emitting unit 16 has several light emitting devices, such as LEDs or OLEDs, to emit rays of different frequencies, or different colors. The control circuit 222 respectively controls the intensity and/or the frequencies (colors) of the rays of each light emitting device. In an embodiment, the light emitting unit 16 has several light emitting devices to emit rays of the same frequency (color), and the control circuit 222 respectively controls the intensity of the rays of each light emitting device only.

FIG. 4 shows a light acupuncture module 3 of the third preferred embodiment of the present invention, which is similar to the first embodiment, except that a solar panel 24 is provided instead of the lid 20. The driving unit has a thin film battery 26. The thin film battery 26 usually has an integrated control circuit and battery to make the light acupuncture module 3 smaller. The solar panel 24 is electrically connected to the control circuit of the thin film battery 26. The solar panel 24 generates electricity from solar power to supply the light emitting unit 16 with electricity. The solar panel 24 charges the thin film battery 26 also, so that the light acupuncture module 3 still may work in a dark environment.

In an embodiment, the light emitting unit 16 has at least an organic light emitting diode (OLED) while the driving unit is the thin film battery. The OLED may be mounted on a substrate of the thin film battery 26 directly to make the stimulating module 10 smaller and to integrate the light emitting unit and the driving unit together. The thin film battery may be a Lithium battery, a printed battery, an organic radical battery, a solar cell, a fuel cell, and so on.

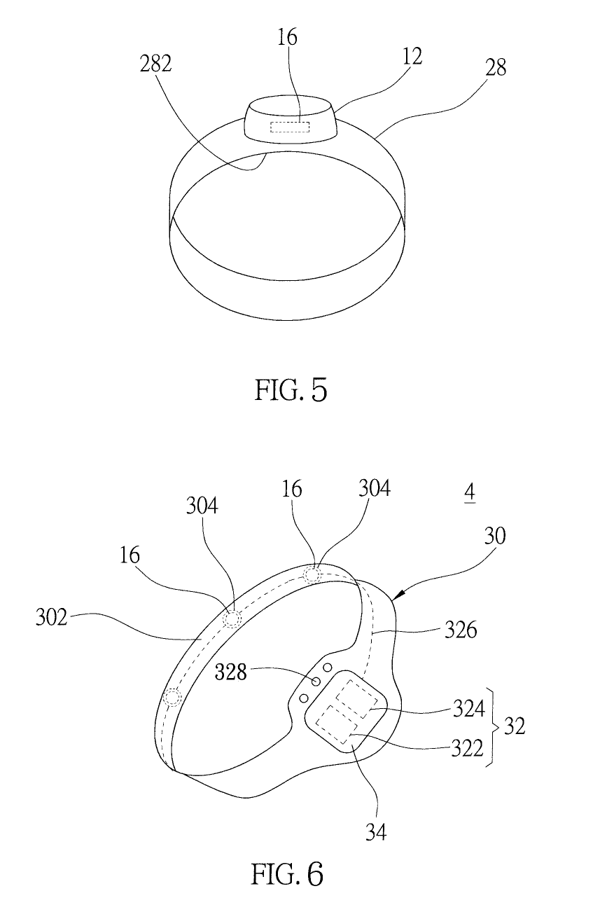

FIG. 5 shows the base 12 and a positioning device, which is a belt 28 adapted to be worn on human's body. An interior side of the belt 28 is a touching face to touch human's skin and let the rays of the light emitting unit 16 emits to an acupuncture point. The belt 28 would have different sizes and shapes for different people.

FIG. 6 shows a light acupuncture module 4 of the fourth preferred embodiment of the present invention, which is similar to the embodiments of above, except that the main member is a wrist band 30. The wrist band 30 has several recesses 304, and a plurality of light emitting units 16 are received in the recesses 304. An interior side 302 of the wrist band 30 is a touching face to touch human's skin and let the rays of the light emitting units 16 emit to acupuncture points respectively. The wrist band 30 further has a chamber 34 to receive a driving unit 32 therein. The driving unit 32 has a battery 322 and a control circuit 324. The battery 322 is electrically connected to the control circuit 324, and the control circuit 324 is electrically connected to the light emitting units 16 through a wire 326. The wrist band 30 is provided with several control keys 328, which are electrically connected to the control circuit 324 for manipulation to change the intensities, frequencies, and/or colors of the rays of the light emitting units 16. The light acupuncture device 4 of the fourth preferred embodiment has the same function as above.

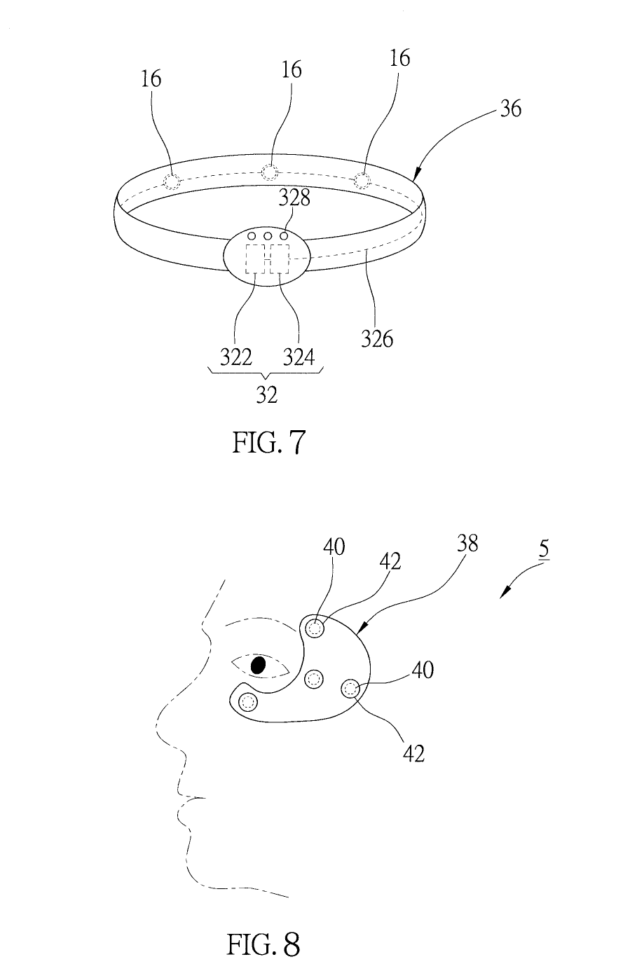

FIG. 7 shows the main member is a belt 36. The rest elements are the same as above.

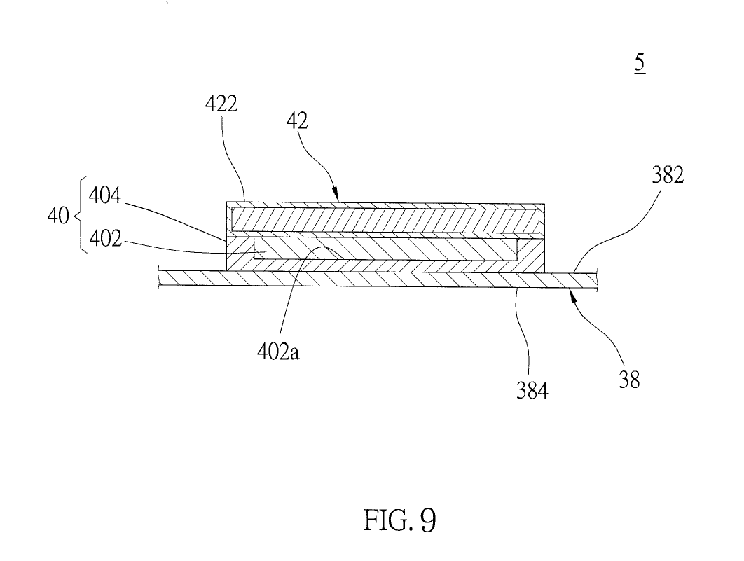

FIG. 8 and FIG. 9 show a light acupuncture module 5 of the fifth preferred embodiment of the present invention, which is similar to the embodiments of above, except that the main member is an adhesive member, such as an adhesive plate 38. In an embodiment, the adhesive plate 38 is made of transparent silicon rubber and has a specific shape for a special portion of human body. The adhesive plate 38 has a top side 382 and a bottom side 384. The bottom side 384 is an adhering face to be attached to skin. A plurality of light emitting units 40 are mounted on the top side 382 of the adhesive plate 38. Each light emitting unit 40 has an OLED 402 and a package member 404. A light emitting face 402a of the light emitting unit 40 faces the top side 382 of the adhesive plate 38. The package member 404 embeds the OLED 402 therein to isolate the OLED 402 from moisture. The driving unit has a plurality of thin film batteries 42 to be electrically connected to the light emitting units 40. Each light emitting units 40 has a protective layer 422 to isolate moisture. The thin film batteries 42 supply the OLEDs 402 of the light emitting units 40 with electricity and control their intensity. The adhesive plate 38 is attached to skin to let the rays of the OLEDs 402 emit through the adhesive plate 38 and stimulate specific acupuncture points.

Because the light emitting units 40 utilize the OLEDs 402, the OLEDs 402 and the thin film batteries 42 may share the same substrate, which means that the OLED 402 may be mounted on the substrate of the thin film battery 42, and covered by the protective layer 422. The size of the light acupuncture module 5 will be very small, so that the light acupuncture module 5 is disposable. In an embodiment, the light emitting unit 40 may utilize LED, and the LED chip is mounted on the substrate of the thin film battery 42 and covered by the protective layer 422.

The main member may be provided with a magnetic member, such as magnet, to generate a magnetic field. Therefore, the light acupuncture module of the present invention may stimulate the acupuncture point with light and magnetic field.

The light acupuncture module of the present invention may be attached to human skin directly to let the user get light acupuncture therapy. With the light acupuncture module of the present invention, one may get light acupuncture therapy anytime and anywhere, and he/she can do anything he/she likes without any restriction.

It must be pointed out that the embodiments described above are only some preferred embodiments of the present invention. All equivalent structures which employ the concepts disclosed in this specification and the appended claims should fall within the scope of the present invention.

What is claimed is:

1. A light acupuncture module, comprising:
a main member having a touching face adapted to touch a human's skin;
a light emitting unit, which is connected to the main member, having a light emitting face, from which specific rays emits; and
a driving unit electrically connected to the light emitting unit to supply the light emitting unit with electricity.

2. The light acupuncture module of claim 1, wherein the main member has a base and a positioning device; the base has a room therein; the positioning device is provided on a bottom side of the base; the touching face is on the positioning device; and the light emitting unit and the driving unit are received in the room of the base.

3. The light acupuncture module of claim 2, wherein the positioning device is an adhesive layer.

4. The light acupuncture module of claim 2, wherein the driving unit has a control circuit and a battery; the battery is electrically connected to the control circuit, and the control circuit is electrically connected to the light emitting unit to control an intensity of the rays of the light emitting unit.

5. The light acupuncture module of claim 4, wherein the driving unit is a thin film battery.

6. The light acupuncture module of claim 4, wherein the light emitting unit has at least a light emitting diode or at least an organic light emitting diode.

7. The light acupuncture module of claim 4, wherein the light emitting unit has a plurality of light emitting devices to emit the rays of the same frequency or different frequencies.

8. The light acupuncture module of claim 7, wherein the control circuit changes intensities of the rays of the light emitting devices respectively.

9. The light acupuncture module of claim 7, wherein the light emitting devices are light emitting diodes or organic light emitting diodes.

10. The light acupuncture module of claim 2, further comprising a solar panel connected to the base, wherein the base has a top opening communicated with the room, and the solar panel closes the top opening and is electrically connected to the driving unit.

11. The light acupuncture module of claim 2, further comprising a lid connected to the base, wherein the base has a top opening communicated with the room, and the lid closes the top opening.

12. The light acupuncture module of claim 1, wherein the main member has a recess to receive the light emitting unit and a chamber to receive the driving unit.

13. The light acupuncture module of claim 12, wherein the driving unit has a control circuit and a battery; the battery is electrically connected to the control circuit, and the control circuit is electrically connected to the light emitting unit to control an intensity of the rays of the light emitting unit.

14. The light acupuncture module of claim 13, wherein the light emitting unit has a plurality of light emitting devices to emit the rays of different frequencies.

15. The light acupuncture module of claim 14, wherein the control circuit changes intensities of the rays of the light emitting devices respectively.

16. The light acupuncture module of claim 15, wherein the light emitting devices are light emitting diodes or organic light emitting diodes.

17. The light acupuncture module of claim 1, wherein the main member is an adhesive member, which has an adhering face to form the touching face; the light emitting unit is connected to the adhesive member; and the rays of the light emitting unit emits through the adhesive member.

18. The light acupuncture module of claim 17, wherein the adhesive member is an adhesive plate.

19. The light acupuncture module of claim 17, wherein the driving unit is a thin film battery.

20. The light acupuncture module of claim 19, wherein the light emitting unit has at least an organic light emitting diode and a package member to embed the organic light emitting diode therein.

21. The light acupuncture module of claim 20, wherein the thin film battery changes an intensity of the rays of the organic light emitting diode.

22. The light acupuncture module of claim 12, further comprising a magnetic member on the main member.
